(19) United States
(12) Patent Application Publication
(10) Pub. No.: US 2003/0162215 A1
(43) Pub. Date: Aug. 28, 2003

(54) Method for detecting biopolymers

(75) Inventors: Kanako Iwao, Tokyo (JP); Motonao Nakao, Tokyo (JP); Keiichi Sato, Tokyo (JP); Toshiki Morita, Tokyo (JP); Susumu Kuwabata, Osaka (JP)

Correspondence Address: Reed Smith Hazel & Thomas LLP, Suite 1400, 3110 Fairview Park Drive, Falls Church, VA, 22042-4503, US

(73) Assignee: Hitachi Software Engineering Co., Ltd.

(21) Appl. No.: 10/372,808

(22) Filed: Feb. 26, 2003

(30) Foreign Application Priority Data
Feb. 27, 2002 (JP) 2002-51532
Feb. 25, 2003 (JP) 2003-47413

(51) Int. Cl. A61L 2/00; B05D 3/00; C12Q 1/68; G01N 33/53

(52) U.S. Cl. 435/6; 435/7.5; 438/1; 427/2.11

(57) ABSTRACT

A technique is provided that easily detects biopolymers such as a DNA or a protein by utilizing semiconductor nanoparticles having different excitation wavelengths and fluorescence due to differences in particle size. By binding the semiconductor nanoparticles with avidin (or biotin), detection of biopolymers labeled with biotin (or avidin) is enabled.

### BACKGROUND OF THE INVENTION

1. Technical Field

The present invention relates to a technique in which a semiconductor nanoparticle is bound to a molecule for detection, such as avidin, streptavidin or biotin, for detecting, as a fluorescent substance, a biopolymer such as a polynucleotide or a protein or the like.

2. Background Art

Conventionally, Cy3 and Cy5, fluorescent dyes used with DNA chips, are incorporated as fluorescent substances when performing reverse transcription reaction of RNA. The reaction will now be briefly described (FIG. 1).

First, RT-PCR of mRNA is performed using reverse transcriptase. At this time, Cy3-dUTP or Cy5-dUTP is incorporated and unreacted dUTP is removed to prepare the target cDNA. Next, hybridization of the target cDNA with cDNA on a DNA chip is conducted. Finally, a laser beam is irradiated onto the DNA chip to detect fluorescence with wavelength. A laser beam having an excitation wavelength of 552 nm is irradiated for Cy3, and a laser beam having an excitation wavelength of 650 nm is irradiated for Cy5.

### SUMMARY OF THE INVENTION

However, in the above-described method, Cy3 and Cy5 are individually excited by their respective lasers, and it is only possible to detect one fluorescence with wavelength at a time. In other words, it is only possible to detect the fluorescence with wavelength corresponding to one excitation wavelength.

We found that by using semiconductor nanoparticles in which the particle size has been controlled, it is possible to produce reagents for biopolymer detection bound with molecules for biopolymer detection such as avidin or biotin. Unlike common fluorescent substances such as Cy3 or Cy5, semiconductor nanoparticles can be excited by a single laser beam, to perform detection with a plurality of fluorescence wavelengths by changing the particle size thereof. In the present invention, a biopolymer, that is the object of detection, is not particularly limited, and examples thereof include a protein, a peptide, a polynucleotide such as DNA or RNA, or a saccharide or the like. Moreover, we found that by using the reagent with different particle sizes, it is possible to simultaneously detect a plurality of biopolymers using one excitation wavelength. Furthermore, we found that it is possible to detect trace quantities of a biopolymer by utilizing a crosslinking reaction among semiconductor nanoparticles.

More specifically, the present invention provides the following (1) to (27):

(1) A method for producing a reagent for detecting a biopolymer, comprising the steps of:

(a) preparing a semiconductor nanoparticle having a functional group exposed on its surface by reacting the semiconductor nanoparticle with a substituted alkylthiol; and

(b) binding the semiconductor nanoparticle having a functional group exposed on its surface with a molecule for detection via the above functional group.

(2) The method of (1) above, wherein the reaction is a substitution reaction.

(3) The method of (1) or (2) above, wherein the substituted alkylthiol is an alkylthiol compound having a functional group selected from the group consisting of an amino group, a carboxyl group and a sulfonic acid group.

(4) The method of any of (1) to (3) above, wherein the molecule for detection is avidin or streptavidin, or biotin.

(5) The method of (4) above, wherein, after a semiconductor nanoparticle having a carboxyl group exposed on its surface is derivatized, it is reacted with aminated avidin or streptavidin.

(6) The method of (4) above, wherein a semiconductor nanoparticle having an amino group exposed on its surface is reacted with a derivatized biotin.

(7) The method of any of (1) to (6) above, wherein 1 to 1000 molecules for detection are bonded to every 1 semiconductor nanoparticle.

(8) The method of any of (1) to (7) above, wherein the binding of molecule for detection onto the semiconductor nanoparticle is controlled by adjusting the proportions of several kinds of substituted alkylthiols.

(9) A reagent for detecting a biopolymer obtained by the method according to any of (1) to (8) above.

(10) The reagent of (9) above, wherein the biopolymer is a protein or a polynucleotide.

(11) A method for detecting biopolymers using the reagent according to (9) or (10) above.

(12) The method of (11) above, wherein the method is carried out on a microarray.

(13) The method of (12) above, wherein the microarray is a DNA chip.

(14) The method of (12) above, wherein the microarray is a protein chip.

(15) The method for detecting biopolymers of any of (11) to (14) above, comprising the steps of:

binding a semiconductor nanoparticle with avidin or streptavidin, and

detecting a biotin-labeled biopolymer by means of the fluorescence of the semiconductor nanoparticle.

(16) The method of (15) above, wherein, after an oligonucleotide immobilized onto a DNA chip and a biotin-labeled oligonucleotide are hybridized, the presence or absence of hybridization is detected by adding thereto a semiconductor nanoparticle bonded with avidin or streptavidin.

(17) The method of (15) above, wherein, after a cDNA immobilized onto a DNA chip and a biotin-labeled cDNA are hybridized, the presence or absence of hybridization is detected by adding thereto a semiconductor nanoparticle bonded with avidin or streptavidin.

(18) The method of (15) above, wherein, after an oligonucleotide immobilized onto a DNA chip and a biotin-labeled cDNA are hybridized, the presence or absence of hybridization is detected by adding thereto a semiconductor nanoparticle bonded with avidin or streptavidin.

(19) The method of (15) above, wherein, after a protein immobilized on a protein chip and a biotin-labeled protein are bonded, the presence or absence of bonding between the proteins is detected by adding thereto a semiconductor nanoparticle bonded with avidin or streptavidin.

(20) The method of any of (11) to (14) above, comprising the step of:

binding a semiconductor nanoparticle with biotin, and

detecting a biopolymer labeled with avidin or streptavidin by means of the fluorescence of the semiconductor nanoparticle.

(21) The method according to (20) above, wherein, after an oligonucleotide immobilized onto a DNA chip and an oligonucleotide labeled with avidin or streptavidin are hybridized, the presence or absence of hybridization is detected by adding thereto a semiconductor nanoparticle bonded with biotin.

(22) The method according to (20) above, wherein, after a cDNA immobilized onto a DNA chip and a cDNA labeled with avidin or streptavidin are hybridized, the presence or absence of hybridization is detected by adding thereto a semiconductor nanoparticle bonded with biotin.

(23) The method according to (20) above, wherein, after an oligonucleotide immobilized onto a DNA chip and a cDNA labeled with avidin or streptavidin are hybridized, the presence or absence of hybridization is detected by adding thereto a semiconductor nanoparticle bonded with biotin.

(24) The method of (20) above, wherein, after a protein immobilized on a protein chip and a protein labeled with avidin or streptavidin are bonded, the presence or absence of bonding between the proteins is detected by adding thereto a semiconductor nanoparticle bonded with biotin.

(25) The method of any of (11) to (24) above, wherein the particle size of the semiconductor nanoparticle is within the range of 2 to 10 nm.

(26) The method of any of (11) to (25) above, wherein a plurality kinds of biopolymers are detected using several kinds of semiconductor nanoparticles of different particle sizes.

(27) The method of any of (11) to (26) above, wherein a plurality of semiconductor nanoparticles having the same particle size are cross-linked to carry out detection.

This specification includes part or all of the contents as disclosed in the specifications and/or drawings of Japanese Patent Application Nos. 2002-51532 and 2003-47413, which are priority documents of the present application.

### BRIEF DESCRIPTION OF THE DRAWINGS

FIG. 1 illustrates one example of an experimental procedure using a DNA chip.

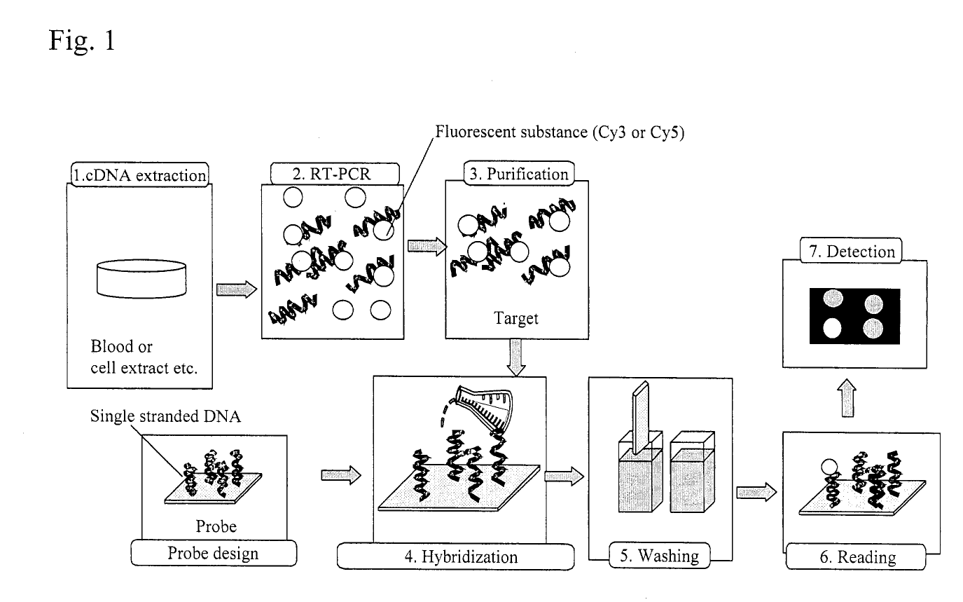

FIG. 2 shows the fluorescence spectrums of CdS particles of a particle size of 2.4 nm and 2.1 nm coated with ZnS employing TOP/TOPO as a stabilizer.

FIG. 3 illustrates one example of a detection procedure by means of a DNA chip using semiconductor nanoparticles.

FIG. 4 illustrates one example of a binding reaction between a semiconductor nanoparticle and avidin.

FIG. 5 illustrates one example of a binding reaction between a semiconductor nanoparticle and biotin.

FIG. 6 illustrates a schematic view of binding between an avidin-bonded semiconductor nanoparticle and a biotin-labeled oligo DNA.

FIG. 7 illustrates a schematic view of self-assembly of semiconductor nanoparticles using disuccinimide.

FIG. 8 illustrates a schematic view of self-assembly of semiconductor nanoparticles using hydroxydisuccinimide.

### DETAILED DESCRIPTION OF THE PREFERRED EMBODIMENTS

Hereinafter, preferred embodiments of the present invention will be described.

There are various kinds of semiconductors, and they can be broadly classified into element semiconductors (silicon, germanium, etc.), oxide semiconductors (cuprous oxide, zinc oxide, etc.), sulfide semiconductors (cadmium sulfide, lead sulfide, zinc sulfide, etc.), compound semiconductors (gallium sulfide, indium phosphide, etc.), and the like. A semiconductor nanoparticle is one in which a semiconductor material such as CdS, ZnS, or CdSe is made into a nano-level microparticle.

When the particle size is about 10 nm or less, a semiconductor nanoparticle emits fluorescence when subjected to photoexcitation. Wavelengths required to excite a semiconductor nanoparticle exist broadly on the ultraviolet side, and the bigger the particle size, the longer wavelength side of the excitation spectrum shifts toward the longer wavelength side. Further, the fluorescence wavelength also shifts to the longer wavelength side in accordance with an increase in the particle size of the semiconductor nanoparticle. Thus, a semiconductor nanoparticle has a characteristic whereby its fluorescence wavelength changes according to its particle size.

In the case of semiconductor, it possesses a characteristic whereby even when light having energy much larger than the energy width of the forbidden band is irradiated thereto, excitation occurs and fluorescence is emitted, and the fluorescence wavelength emitted thereof is identical to the wavelength of fluorescence emitted by irradiating light of about the same energy as the energy width of the forbidden band. Accordingly, when semiconductor nanoparticles of different particle sizes are mixed, by irradiating light having an energy greater than the energy required to excite the semiconductor nanoparticle of the smallest particle size, fluorescences determined by the energy width of the forbidden bands of all the different semiconductor nanoparticles contained therein are emitted at the same time, and by detecting these fluorescences, it is possible to simultaneously detect a plurality of semiconductor nanoparticles of different particle sizes. For example, when semiconductor nanoparticles having particle sizes of 4 nm, 6 nm and 8 nm are excited by excitation light of the same wavelength, specific fluorescence wavelengths that are distinct to each of the nanoparticles are emitted, and by using these fluorescence wavelengths it is possible to simultaneously detect several kinds of biopolymers.

Consequently, by controlling the particle sizes of semiconductor nanoparticles to produce several kinds of semiconductor nanoparticles of different particle sizes, it is possible to easily produce several kinds of fluorescence labeling substances having different fluorescence wavelengths.

Further, using several kinds of semiconductor nanoparticles of different chemical compositions, it is possible to produce several kinds of fluorescence labeling substances having different fluorescence wavelengths and to use these to simultaneously detect several of kinds of biopolymers.

According to the present invention, examples of a useful semiconductor nanoparticle include ZnS, ZnSe, ZnTe, CdS, CdSe, CdTe, InGaAs, and InP semiconductor nanoparticles. Further, according to the present invention, a useful semiconductor nanoparticle includes not only a semiconductor nanoparticle made of one kind of semiconductor, but also includes a semiconductor nanoparticle made of one kind of semiconductor coated by another kind of semiconductor having a wider band gap. For example, the fluorescence intensity of CdS by itself is low, but by coating CdS with ZnS the fluorescence intensity is increased by approximately three-fold and thus, a semiconductor nanoparticle that is more suited for use can be obtained.

The method for producing semiconductor nanoparticles is not particularly limited, as long as particles having particle sizes of 2 to 10 nm are obtained. Examples of a method of synthesizing semiconductor nanoparticles include, for example, the so-called high temperature method described in Z. A. Peng et al., J. Am. Chem. Soc. 2001, 123, 183-184, and the photo-etching technique developed by the present inventors and others (Japanese Patent Application No. 2001-210902; T. Trimoto et al., J. Electrochem. Soc., Vol. 145, No. 6, June 1998; H. Matsumoto et al., Chemistry Letters 595-596, 1995 and the like).

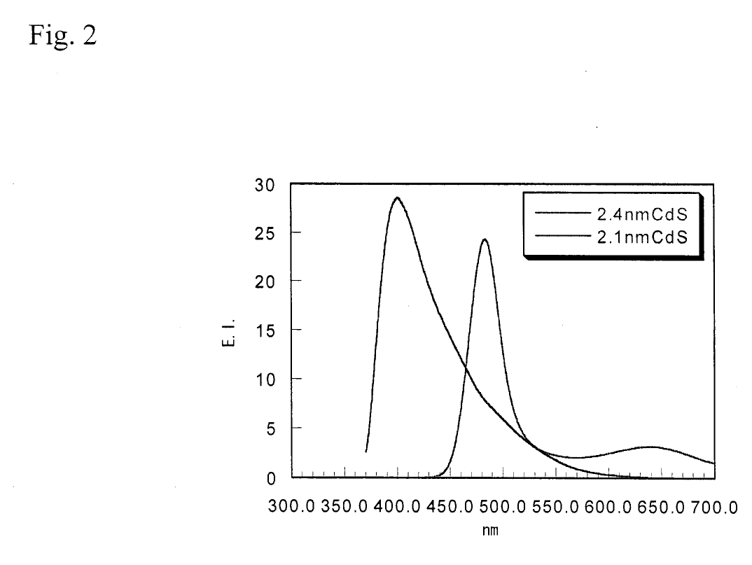

FIG. 2 shows the fluorescence spectrums of semiconductor nanoparticles produced by the photo-etching technique (stabilizer: HMP) of 2.4 nm and 2.1 nm sized CdS particles coated with ZnS. This data was obtained for a case where TOP/TOPO was substituted as the stabilizer during ZnS coating. As is clear from the figure, a difference in particle size of a mere 0.3 nm results in markedly different fluorescence spectrums. By utilizing this characteristic in the present invention, simultaneous detection of a plurality of targets is enabled.

Subsequently, by reacting the obtained semiconductor nanoparticles with substituted alkylthiol, semiconductor nanoparticles having functional groups exposed on its surface can be easily produced. For example, when using CdS semiconductor nanoparticles, by adding the CdS semiconductor nanoparticles to a solution containing substituted alkylthiol compound (HS-R) and stirring, a substitution reaction occurs between the S of the CdS and the S of the thiol compound, and the thiol compound covalently bonds (Cd—S—R) to the surface to modify the entire particle surface with thiol. This reaction is a substitution reaction that can easily proceed by mixing the semiconductor nanoparticles and substituted alkylthiol and stirring. The reaction conditions are not particularly limited, and for example, a product of interest can be easily obtained by stirring at room temperature for 1 hour to 1 day. A substituted alkylthiol is not particularly limited, and a substance having various functional groups substituted at the alkyl group terminus can be used. Preferable examples according to the present invention include an alkylthiol compound having functional groups such as an amino group, a carboxyl group and a sulfonic acid group, and the substance can be suitably selected according to the kind of the molecules for detection to be bonded thereafter.

The number of molecules for detection that can be bound to a single semiconductor nanoparticle can be determined by adjusting the proportions of several kinds of substituted alkylthiol to control the number of reactive functional groups present on the surface of the semiconductor nanoparticle, that is, the number of functional groups that can react at the time of a subsequent binding reaction. By using a thiol compound as an agent to modify the surface of the nanoparticles, it is possible to obtain semiconductor nanoparticles for which the quantity of carboxyl groups or amino groups introduced for every 1 semiconductor nanoparticle is controlled. Examples of a usable thiol compound include 2-mercaptoethanesulfonic acid, 2-aminoethanethiol, 2-mercaptopropionic acid, and 11-mercaptoundecanoic acid.

In the present invention, the number of molecules for detection bonded to every 1 semiconductor nanoparticle is preferably 1 to 1000, and more preferably about 100.

A molecule for detection is not particularly limited as long as it can be used for specifically detecting biopolymers, and examples thereof include avidin or streptavidin, or biotin, an antigen or an antibody, and a DNA or RNA oligonucleotide or polynucleotide or the like.

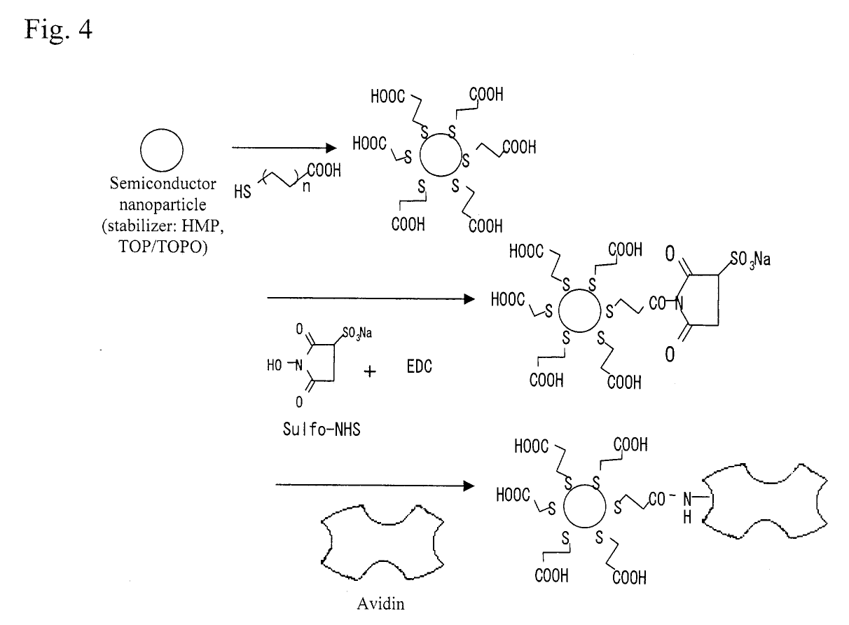

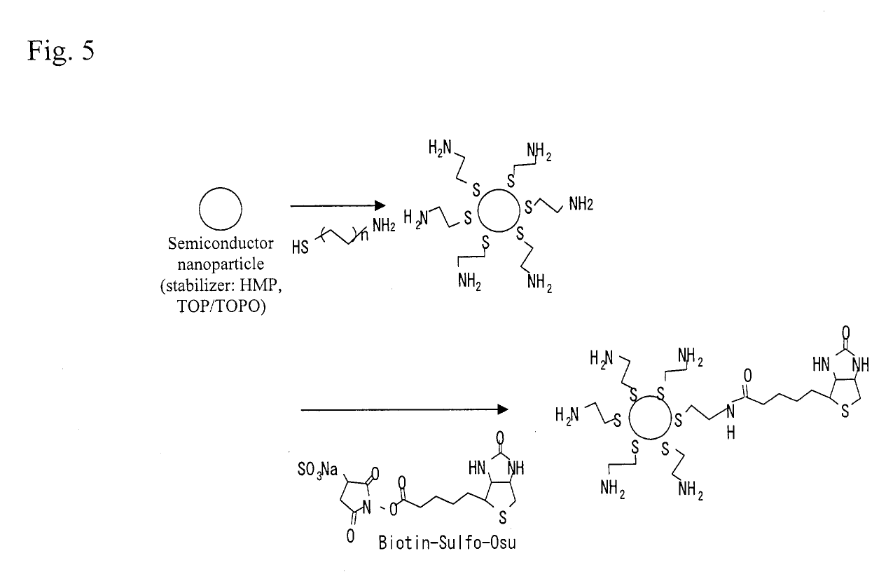

Accordingly, for example in the case of bonding using avidin or streptavidin as a molecule for detection, for example, an alkylthiol compound having a carboxyl group (hereinafter, also referred to as thiolcarboxylic acid) is used as substituted alkylthiol, and bonding can be performed by preparing a semiconductor nanoparticle having a carboxyl group exposed on its surface, and after further derivatization using, for example, N-hydroxysulfosuccinimide or the like, reacting this with avidin or streptavidin (commercially available, for example, from Sigma Aldridge Japan) (FIG. 4). Further, in the case of bonding using biotin as a molecule for detection, for example, an alkylthiol compound having an amino group (hereinafter, also referred to as aminothiol) is used as a substituted alkylthiol, and bonding can be performed by preparing a semiconductor nanoparticle having an amino group exposed on its surface, and then reacting this with derivatized biotin, for example, Biotin-Sulfo-Osu (sulfosuccinimidyl D-biotin) (DOJINDO LABORATORIES) (FIG. 5). A person skilled in the art can appropriately select substitution reaction conditions and reagents suitable for the bonding process according to the kind of functional group on the semiconductor nanoparticle, molecule for detection of interest, and the like. Similar to the above reaction, this substitution reaction can easily proceed by mixing semiconductor nanoparticles with functional groups exposed on their surface with the molecules for detection and stirring. The reaction conditions are not particularly limited, and for example, a product of interest can be easily obtained by stirring at room temperature for 1 hour to 1 day.

Detection of biopolymers using the present invention can be performed by adding the reagent for biopolymer detection according to the present invention to a sample containing a biopolymer, for example, a polynucleotide or protein previously labeled with a molecule capable of specifically reacting with the molecule for detection, isolating semiconductor nanoparticles for which specific binding has occurred, and detecting the fluorescence thereof Binding reaction and detection can also be performed in a solution. Detection may also be performed in a cell containing a biopolymer, and reaction may also be performed on a microarray such as a DNA chip or protein chip.

In an example of one embodiment of the method of the present invention, after hybridizing an oligonucleotide immobilized on a DNA chip with a biotin-labeled oligonucleotide, semiconductor nanoparticles bonded with avidin or streptavidin are added thereto to enable detection of the presence or absence of hybridization. Depending on the presence or absence of hybridization, it is possible to determine whether or not the oligonucleotide of interest is present in a sample. The term “oligonucleotide” used herein includes, but not limited to, a DNA or RNA oligonucleotide having 100 or shorter base length, and it may be of natural origin or may be synthesized.

Further, after hybridizing a cDNA immobilized on a DNA chip with a biotin-labeled cDNA, semiconductor nanoparticles bonded with avidin or streptavidin are added thereto to enable detection of the presence or absence of hybridization. Depending on the presence or absence of hybridization, it is possible to determine whether or not the oligonucleotide of interest is present in a sample.

Moreover, after an oligonucleotide immobilized onto a DNA chip and a biotin-labeled cDNA are hybridized, the presence or absence of hybridization may be detected by adding thereto a semiconductor nanoparticle bonded with avidin or streptavidin. As with the above case, whether or not the oligonucleotide of interest is present in a sample can then be determined depending on the presence or absence of hybridization.

In an example of other embodiment of the method of the present invention, after hybridizing an oligonucleotide immobilized on a DNA chip with an avidin-labeled oligonucleotide, the presence or absence of hybridization can be detected by adding semiconductor nanoparticles bonded with biotin are added thereto to enable detection of the presence or absence of hybridization. Depending on the presence or absence of hybridization, it is possible to determine whether or not the oligonucleotide of interest is present in a sample.

Further, after hybridizing a cDNA immobilized on a DNA chip with an avidin-labeled cDNA, the presence or absence of hybridization can be detected by adding semiconductor nanoparticles bonded with biotin are added thereto enable detection of the presence or absence of hybridization. Depending on the presence or absence of hybridization, it is possible to determine whether or not the oligonucleotide of interest is present in a sample.

Moreover, after an oligonucleotide immobilized onto a DNA chip and a cDNA labeled with avidin or streptavidin are hybridized, the presence or absence of hybridization may be detected by adding thereto a semiconductor nanoparticle bonded with biotin. As with the above case, whether or not the oligonucleotide of interest is present in a sample can then be determined depending on the presence or absence of hybridization.

On the other hand, when detecting a protein, for example, after bonding a protein immobilized on a protein chip with a biotin-labeled protein, the presence or absence of bonding between the proteins can be detected by adding semiconductor nanoparticles bonded with avidin or streptavidin thereto.

Further, after bonding a protein immobilized on a protein chip with a protein labeled with avidin or streptavidin, the presence or absence of bonding between the proteins can be detected by adding semiconductor nanoparticles bonded with biotin thereto.

As described above, according to the method of the present invention several kinds of biopolymers can be detected by using several kinds of semiconductor nanoparticles of different particle sizes or chemical compositions. As long as each peak of the fluorescence spectra of the semiconductor nanoparticles used can be distinguished, several kinds of biopolymers can be detected at the same time, and while also depending on the sharpness of the peaks, for example, two peaks separated by about 100 nm can be adequately distinguished. The detectable range is from 400 nm to 700 nm.

### EXAMPLES

Hereinafter, a technique is described for labeling and detecting a biopolymer by bonding a thiol group (—SH) modified semiconductor nanoparticle with avidin or biotin.

### Example 1

### Bonding Between a Semiconductor Nanoparticle and Avidin or Biotin

A system using an avidin-biotin complex is widely utilized in the fields of tissue staining and immunoassay such as EIA (enzyme immunoassay). Avidin has a high affinity (10¹⁵M⁻¹) to biotin, and it is possible to label a protein, antibody, enzyme or the like with biotin without destroying the activity thereof By subjecting biotin itself to chemical modification, it is possible to bind it to various functional groups of avidin.

FIG. 4 illustrates one example of synthesis of a semiconductor nanoparticle modified with avidin. This reaction involves a two step synthesis, and the following three methods may be mentioned regarding control of the number of avidin molecules modifying on the surface of the nanoparticles:

(1) when modifying with thiolcarboxylic acid, the number of carboxylic acid molecules is controlled by modifying with a mixture of a suitable thiolcarboxylic acid and thiol with an aqueous substituent;

(2) control by means of the amount of N-hydroxysulfosuccinimide mixed in; and

(3) control by means of the number of avidin molecules mixed in.

On the other hand, FIG. 5 illustrates an example of the synthesis of a semiconductor nanoparticle modified with biotin. The semiconductor nanoparticle is modified at its surface using aminothiol. An amino group of this surface-modifying agent is modified with biotin for labelling an amino group (spacer may be any length). For example, semiconductor nanoparticles bonded with biotin can be obtained by first introducing a thiol compound into a semiconductor nanoparticle suspension and stirring overnight under nitrogen atmosphere, and then directly introducing N-hydroxysulfosuccinimide, of an amount equivalent to the amino groups of the thiol compound, into the reaction solution and stirring the resulting solution for 1 hour under nitrogen atmosphere.

In this example, the following methods for controlling the number of biotin molecules modifying the surface of the nanoparticles can be noted:

(1) when modifying with aminothiol, a method in which a suitable aminothiol and a thiol having an aqueous group are mixed to modify biotin, wherein the amount of aminothiol is controlled by means of the mixing ratio; and

(2) a method in which control is carried out by means of the mixing ratio of the biotin for labelling the amino groups and the semiconductor nanoparticles modified with amino groups.

### Example 2

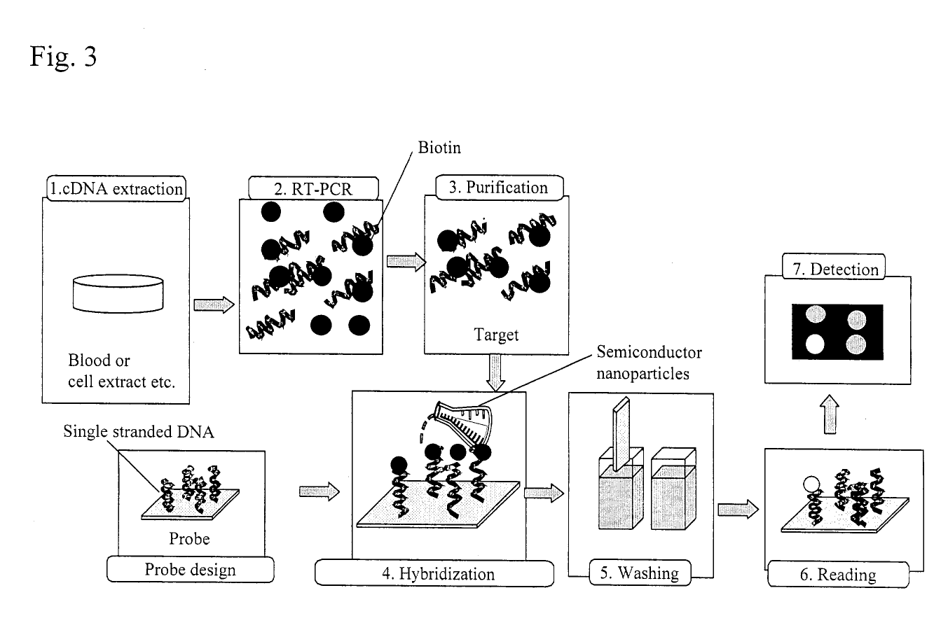

### Hybridization Reaction Using DNA (Target) Labeled Utilizing an Avidin-Biotin System (Detection of DNA on a Chip) (FIG. 3)

In this example, DNA having a terminus modified with biotin or avidin was used. Semiconductor nanoparticles were modified, with avidin or biotin (FIGS. 4 and 5), to serve as DNA fluorescent labels.

1 mRNA Extraction

Ten ml of solution D (guanidine thiocyanate, n-lauryl sarcosine, 1 M sodium citrate, β-mercaptoethanol) was added per 1 g of tissue for homogenization, and sodium acetate (2M, pH 4.0), acid phenol, and chloroform were respectively mixed in by stirring. After cooling on ice for 15 min, the mixture was centrifuged for 30 min at 15000 rpm. An equivalent amount of isopropanol was added to the aqueous layer, and after cooling at −20° C. for 1 hour, washing was performed with 70% ethanol. The mixture was centrifuged at 4° C. for 15 min at 15000 rpm, resuspended in 4 ml of DEPC-treated water and 650 μl of 5 M sodium chloride was added thereto. Eight ml of CTAB/urea solution was then added, and the resultant mixture was centrifuged at room temperature for 15 min at 15000 rpm. Eight ml of ethanol was added thereto and the mixture was cooled at −20° C. for 1 hour, and then centrifuged at 4° C. for 15 min at 15000 rpm. The mixture was washed with 70% ethanol and resuspended in DEPC-treated water.

2 RT-PCR

DEPC-treated water was added to poly(A)-RNA and biotinylated oligo(dT) primer, and the mixture was incubated at 70° C. for 10 min, and then quenched on ice. The following sample (RNA sample/primer mixture, 10×PCR buffer, 25 mM MgCl₂, 10 mM dNTP mix, 0.1 M DTT) was added thereto, and 1 μl of reverse transcriptase was further added thereto. The resulting mixture was incubated at 42° C. for 50 min. Incubation at 70° C. for 50 min was carried out to terminate the reaction, and 1 μl of RNase H was added thereto. This mixture was then incubated at 37° C. for 20 mm to perform PCR, thereby obtaining biotinylated cDNA.

3 Hybridization

20×SSC, ion exchanged water and the above biotinylated cDNA were added into a tube, and incubated at 95° C. for 3 min to denature the DNA, and then 10% SDS was further added. This hybridization solution was poured onto a DNA chip, a cover glass was placed thereon, and incubation at 65° C. for 10-20 hours was carried out. After hybridization, the slide glass was immersed into a 2×SSC 0.1% SDS solution and the cover glass was detached. Washing with SSC was repeated, and then it was centrifuged at 1000 rpm for 2 min, and dried at room temperature.

4 Labelling by Semiconductor Nanoparticles

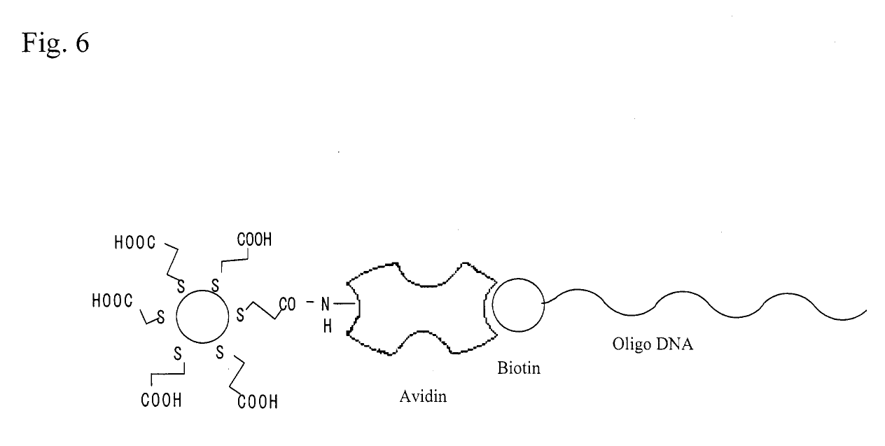

Semiconductor nanoparticles bonded with avidin (FIG. 4) were added to the DNA chip after the hybridization reaction. Reaction was carried out so that only the hybridized cDNA was labeled (FIG. 6). The fluorescence of each spot on the DNA chip was measured using a fluorescence scanner.

### Example 3

### Enhancement of Detection Sensitivity by Self-Assembly of Semiconductor Nanoparticles

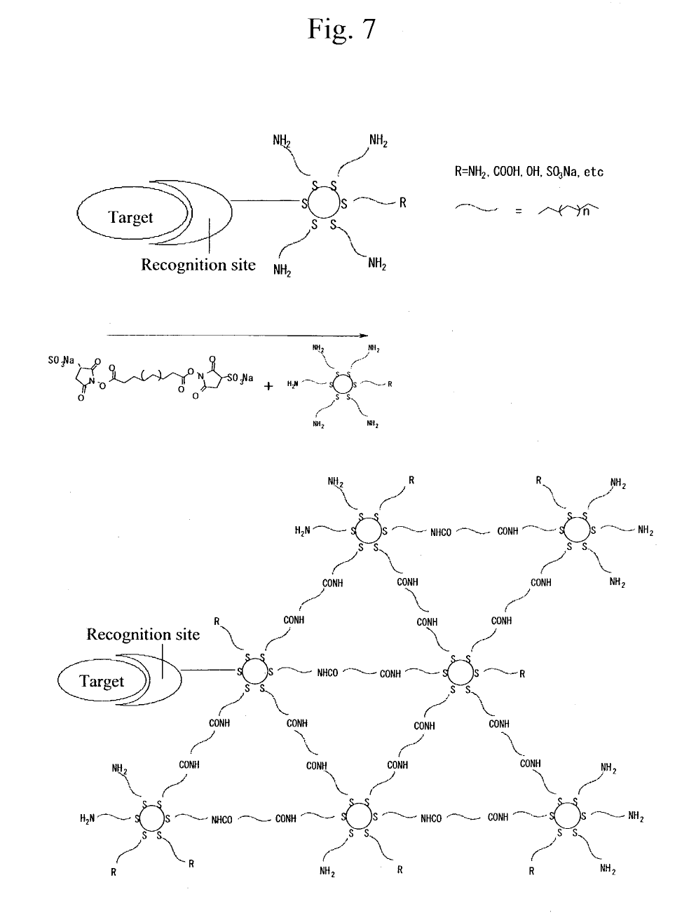

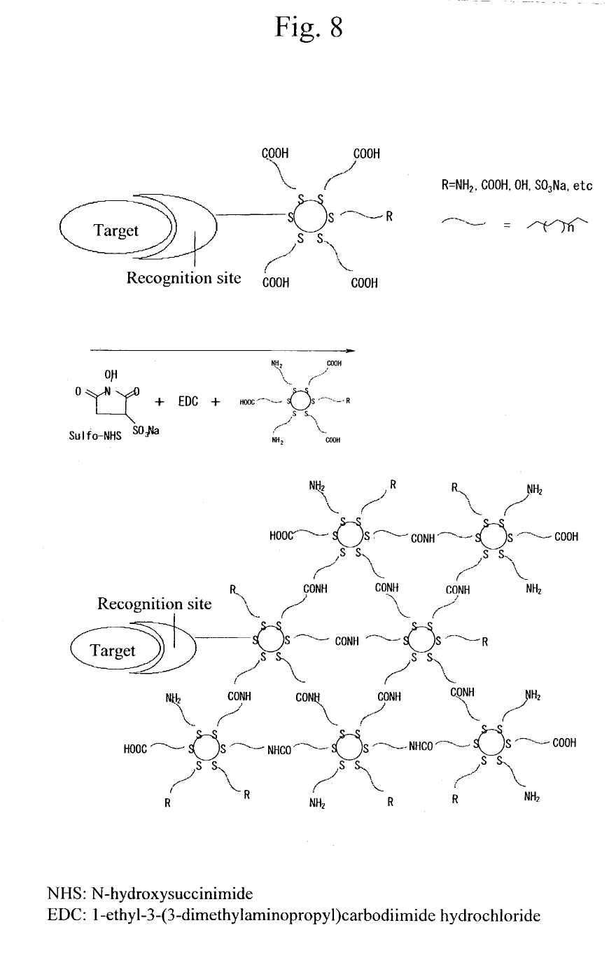

When detecting for biopolymers using semiconductor nanoparticles that have been surface-modified with a thiol compound having an amino group (—NH₂) or carboxyl group (—COOH), the target substance to be detected and the functional group of the semiconductor nanoparticles serving as a label are usually bonded one-to-one. Therefore, detection sensitivity depends on the concentration of the target substance. However, by allowing self-assembly (crosslinking) of a large number of semiconductor nanoparticles with semiconductor nanoparticles labeled to the target substance, it is possible to detect the target substance with high sensitivity without depending on the concentration of the target substance. The binding between the target substance and the recognition site may be, for example, the binding between biotin and avidin, between an oligonucleotide and the complementary strand thereof, between a DNA and a DNA binding protein, between an antibody and an antigen, and the like. Functional groups of a biotin-labeled semiconductor nanoparticle start out by binding one-to-one to a target substance. In the case of an avidin-labeled semiconductor nanoparticle, binding to a target substance takes the form of a 1-to-1 to 1-to-4 relationship. Prior to binding with a target substance, the semiconductor nanoparticle for labeling is surface-modified with a thiol compound having an amino group or carboxyl group. Self-assembly of the semiconductor nanoparticles occurs by means of amide bonding, as illustrated in FIGS. 7 and 8, by introducing thereto: a compound having a succinimidyl group, for example, disuccinimide or hydroxysuccinimide; and the same type of semiconductor nanoparticles with the same particle size as the semiconductor nanoparticles for labeling, having amino groups or carboxyl groups on their surface. By binding a target substance thereto, fluorescence intensity increases and detection sensitivity is noticeably enhanced.

According to the present invention, by using semiconductor nanoparticles for which the particle size has been controlled, a plurality kinds of biopolymers can be simultaneously detected with a single excitation wavelength. Further, while conventional oligonucleotide labelling requires a reverse transcription reaction to incorporate fluorescent substances into cDNA by using an avidin-biotin system at the time of incorporation of the fluorescent substance, this is not required in the method of the present invention. Further, labelling of biopolymers using semiconductor nanoparticles is also enabled.

According to the present invention, by binding avidin or biotin to the semiconductor nanoparticles, it is possible to carry out detection by simply stirring in DNA or protein labeled with avidin or biotin.

Moreover, by binding to a target a semiconductor nanoparticle cross-linked with a large number of semiconductor nanoparticles, high sensitivity detection of a target substance that is not dependent on the concentration of the target substance is enabled. Thus, detection of trace amounts of a target substance is also enabled, even in the case where detection is difficult due to the low fluorescence intensity of one semiconductor nanoparticle molecule.

All publications, patents and patent applications cited herein are incorporated herein by reference in their entirety.

What is claimed is:

1. A method for producing a reagent for detecting a biopolymer, comprising the steps of:
(a) preparing a semiconductor nanoparticle having a functional group exposed on its surface by reacting the semiconductor nanoparticle with a substituted alkylthiol; and
(b) binding the semiconductor nanoparticle having a functional group exposed on its surface with a molecule for detection via said functional group.

2. The method according to claim 1, wherein the reaction is a substitution reaction.

3. The method according to claim 1, wherein the substituted alkylthiol is an alkylthiol compound having a functional group selected from the group consisting of an amino group, a carboxyl group and a sulfonic acid group.

4. The method according to claim 1, wherein the molecule for detection is avidin or streptavidin, or biotin.

5. The method according to claim 4, wherein, after a semiconductor nanoparticle having a carboxyl group exposed on its surface is derivatized, it is reacted with aminated avidin or streptavidin.

6. The method according to claim 4, wherein a semiconductor nanoparticle having an amino group exposed on its surface is reacted with a derivatized biotin.

7. The method according to claim 1, wherein 1 to 1000 molecules for detection are bonded to every 1 semiconductor nanoparticle.

8. The method according to claim 1, wherein the bonding of the molecule for detection onto the semiconductor nanoparticle is controlled by adjusting the proportions of several kinds of substituted alkylthiols.

9. A reagent for detecting a biopolymer obtained by the method according to claim 1.

10. The reagent according to claim 9, wherein the biopolymer is a protein or a polynucleotide.

11. A method for detecting biopolymers using the reagent according to claim 9.

12. The method according to claim 11, wherein the method is carried out on a microarray.

13. The method according to claim 12, wherein the microarray is a DNA chip.

14. The method according to claim 12, wherein the microarray is a protein chip.

15. The method for detecting biopolymers according to claim 11, comprising the steps of:
binding a semiconductor nanoparticle with avidin or streptavidin, and
detecting a biotin-labeled biopolymer by means of the fluorescence of the semiconductor nanoparticle.

16. The method according to claim 15, wherein, after an oligonucleotide immobilized onto a DNA chip and a biotin-labeled oligonucleotide are hybridized, the presence or absence of hybridization is detected by adding thereto a semiconductor nanoparticle bonded with avidin or streptavidin.

17. The method according to claim 15, wherein, after a cDNA immobilized onto a DNA chip and a biotin-labeled cDNA are hybridized, the presence or absence of hybridization is detected by adding thereto a semiconductor nanoparticle bonded with avidin or streptavidin.

18. The method according to claim 15, wherein, after an oligonucleotide immobilized onto a DNA chip and a biotin-labeled cDNA are hybridized, the presence or absence of hybridization is detected by adding thereto a semiconductor nanoparticle bonded with avidin or streptavidin.

19. The method according to claim 15, wherein, after a protein immobilized onto a protein chip and a biotin-labeled protein are bonded, the presence or absence of bonding between the proteins is detected by adding thereto a semiconductor nanoparticle bonded with avidin or streptavidin.

20. The method according to claim 11, comprising the steps of:
binding a semiconductor nanoparticle with biotin, and
detecting a biopolymer labeled with avidin or streptavidin by means of the fluorescence of the semiconductor nanoparticle.

21. The method according to claim 20, wherein, after an oligonucleotide immobilized onto a DNA chip and an oligonucleotide labeled with avidin or streptavidin are hybridized, the presence or absence of hybridization is detected by adding thereto a semiconductor nanoparticle bonded with biotin.

22. The method according to claim 20, wherein, after a cDNA immobilized onto a DNA chip and a cDNA labeled with avidin or streptavidin are hybridized, the presence or absence of hybridization is detected by adding thereto a semiconductor nanoparticle bonded with biotin.

23. The method according to claim 20, wherein, after an oligonucleotide immobilized onto a DNA chip and a cDNA labeled with avidin or streptavidin are hybridized, the presence or absence of hybridization is detected by adding thereto a semiconductor nanoparticle bonded with biotin.

24. The method according to claim 20, wherein, after a protein immobilized on a protein chip and a protein labeled with avidin or streptavidin are bonded, the presence or absence of bonding between the proteins is detected by adding thereto a semiconductor nanoparticle bonded with biotin.

25. The method according to claim 11, wherein the particle size of the semiconductor nanoparticle is within the range of 2 to 10 nm.

26. The method according to claim 11, wherein a plurality kinds of biopolymers are detected using several kinds of semiconductor nanoparticles of different particle sizes.

27. The method according to claim 11, wherein a plurality of semiconductor nanoparticles having the same particle size are cross-inked to carry out detection.
